(19) United States
(12) Patent Application Publication
(10) Pub. No.: US 2009/0124952 A1
(43) Pub. Date: May 14, 2009

(54) Herpes Treatment and Dressing

(76) Inventor: David A. Berman, Los Altos, CA (US)

Correspondence Address: SHAY GLENN LLP, 2755 CAMPUS DRIVE, SUITE 210, SAN MATEO, CA, 94403, US

(21) Appl. No.: 12/267,755

(22) Filed: Nov. 10, 2008

Related U.S. Application Data
(60) Provisional application No. 60/987,017, filed on Nov. 9, 2007.
(60) Provisional application No. 61/131,857, filed on Jun. 11, 2008.

(51) Int. Cl. A61F 13/02

(52) U.S. Cl. 602/54

(57) ABSTRACT

A dressing assembly configured to treat a herpetic lesion including a semi-permeable material sized and shaped to cover a herpetic lesion on human tissue. The invention also provides a method of treating a herpetic lesion (such as lesions caused by, e.g., herpes simplex 1, herpes simplex 2, or herpes zoster) including the step of applying a semi-permeable dressing to cover said wound for a sufficient time to heal said lesion.

### CROSS-REFERENCE TO RELATED APPLICATIONS

This application claims priority to U.S. Provisional Application No. 60/987,017, filed Nov. 9, 2007; and to U.S. Provisional Application No. 61/131,857, filed Jun. 11, 2008, which applications are incorporated by reference as if fully set forth herein.

### BACKGROUND OF THE INVENTION

This invention relates to the treatment of viral lesions, and more particularly to a system and method for dressing herpes lesions, and other viral sores with a herpes dressing.

The herpes viruses comprise a large family of double stranded DNA viruses. These enveloped viruses infect cells that carry negatively charged structures such as heparin sulfate and or glycosaminoglycans, in addition to a herpes viral entry mediator, on their surface. A characteristic feature of these viruses is their ability to remain latent in their host for life after primary infection and to reactivate more or less frequently from a pool of latent infected cells upon diverse internal and external stimuli.

Eight of the herpes viruses, herpes simplex virus types 1 and 2 (HSV-1 and HSV-2), varicella zoster virus (VZV), human cytomegalovirus (HCMV), Epstein-Barr virus (EBV), and human herpes viruses 6, 7, and 8 (HHV-6, HHV-7, and HHV-8), have been shown to infect humans. Several of these viruses are important human pathogens.

Herpes simplex virus (HSV) can cause severe life-threatening infections in immunocompromised patients and in the neonate, while exhibiting a self-limiting disease in an immunocompetent host. Even in immunocompetent humans, HSV can cause embarrassing and painful blisters that take up to four weeks to heal. Further, HSV can cause corneal infection leading to permanent scarring, loss of vision, and blindness in immunocompetent hosts. In fact, HSV is the leading cause of blindness in young adults. Despite the availability of antivirals and steroids to suppress inflammation associated with acute herpetic disease, chronic HSV continues to cause significant ocular morbidity. Accordingly, new ways to limit transmission of HSV are urgently needed.

HSV-1 is estimated to affect 100 million people in the U.S. Primary infection of HSV-1 usually occurs between the ages of one and four. Cold sores, the visible symptom, typically appear at a later age, with most of the population being infected by the age of 20. (Medical Encyclopedia; Herpes labialis). Genital herpes (HSV-2) is the second most common sexually transmitted disease, with approximately 45 million Americans being affected (CDC 2004). One in five adults is estimated to be infected with HSV-2. VZV is the causative agent of chicken pox upon primary infection and can recur in adults as shingles.

Symptoms that manifest during an HSV (type 1 or 2) infection vary in appearance and intensity from patient to patient. Some individuals experience such mild symptoms that they are unaware of their infection. Others experience flu-like symptoms including fever and swollen glands, particularly in the lymph nodes near the neck or groin. Headache and painful urination also sometimes accompany full-blown symptoms of first episodes. Certain symptoms that manifest in a HSV infection may begin approximately two days before lesions appear. These symptoms include: itching, burning, or tingling sensation.

Although the recurring disease may exhibit similar symptoms as the initial infection, often the symptoms are milder. The common symptoms that most people associate with genital herpes are sores, vesicles, or ulcers—all of which can also be called lesions. These lesions of genital herpes often resemble small pimples or blisters that eventually crust over and finally scab like a small cut. These lesions may take anywhere from two to four weeks to heal fully. (ASMA)

The lesions that form from an infection of HSV can appear on the lips, mouth, or gums for oral herpes. For HSV that infects the genital area (genital herpes) wounds can be on the penis and scrotum in men; and on the vaginal area or in the cervix on women. In addition, wounds can be found in the urinary tract, around the anal opening, on the buttocks or thighs, and on other parts of the body.

HSV is transmitted through direct skin-to-skin or mucous membrane contact. This occurs when a contagious area comes into contact with a tiny break in the skin or mucous membrane tissue, primarily the mouth and genitals. The surest way to avoid transmission of HSV is to abstain from any skin-to-skin contact with a contagious area. For genital herpes, this means abstaining from sexual contact. Correct and consistent use of latex condoms can reduce the risk of genital herpes only when the infected area or site of potential exposure is protected. Since a condom may not cover all infected areas, even correct and consistent use of latex condoms cannot guarantee protection from genital herpes. Some method to protect areas not covered by a condom is needed.

Currently there is no cure for HSV. Medications exist that can abbreviate primary infection and reduce the frequency and duration of outbreaks for latent HSV infections. However, anyone infected with HSV can still have recurring episodes of herpes lesions even with the medication. The patient must take the medication at the first sign of the outbreak (e.g., itching, burning, or tingling sensation) to reduce the duration of the outbreak and then the duration is only reduced by one to two days.

Most of the currently available anti-herpes drugs target the viral DNA polymerase. Drugs such as Foscarnet act by direct inhibition of the viral polymerase. These drugs are non-nucleoside inhibitors of DNA polymerases. Others such as the nucleoside analogs, Valacyclovir, Penciclovir and Ganciclovir must first be phosphorylated to the monophosphate forms by virus encoded kinases and, further phosphorylated to triphosphate by cellular enzymes before they are active inhibitors. The triphosphate forms of these nucleoside analogs inhibit polymerases by competing with the binding of natural triphosphates and their subsequent insertion into growing DNA strands. These drugs are known as nucleoside inhibitors of herpes virus DNA polymerases. Side effects associated with these nucleoside analogs drugs include nausea, headache, migraine, fever, vomiting, diarrhea, flatulence, dizziness, abdominal pain, dysmenorrhea, arthralgia, fatigue, skin rash, agitation, vertigo, confusion, sore throat, constipation, edema, renal impairment, and depression. In addition, rare adverse effects (<0.1% of patients) include: coma, seizures, neutropenia, leukopenia, tremor, ataxia, encephalopathy, psychotic symptoms, crystalluria, anorexia, hepatitis, Stevens-Johnson syndrome, toxic epidermal necrolysis and/or anaphylaxis. Therefore, a method to treat herpes wounds without significant side is urgently needed.

Non-pharmaceutical treatments of herpetic lesions include keeping the lesions clean and dry. Patients are often advised to use a hair dryer to dry herpetic lesions.

The present invention provides a method for hastening the healing of HSV lesions and inhibiting HSV transmission from a contagious lesion. The method offers a distinct advantage in the treatment of patients in need, because it employs only a semi-permeable film dressing containing no therapeutic therapy.

### SUMMARY OF THE INVENTION

One aspect of the invention pertains to treatment of herpetic wounds, sores, blisters, or lesions by applying a semi-permeable film or membrane dressing to the affected areas. The treatment of the herpetic wound involves applying the dressing to cover the wound and leaving the dressing on the wound until such time to heal the wound. The semi-permeable film dressing is comprised of an adhesive and backing material. The adhesive may be polyvinyl ethyl ether, (methyl vinyl ether), polymethyl vinyl ether, and polyacrylic ester.

In another embodiment of the invention the backing material is comprised of a copolymer made by copolymerizing a hydroxy alkyl acrylate or methacrylate with an alkoxy alkyl acrylate or methacrylate and optionally with a minor amount of a further monomer. Another embodiment of the invention uses backing material comprised of a homopolymer of ethoxy ethyl methacrylate or methoxy ethyl methacrylate or a copolymer of methyl methacrylate with ethoxy methacrylate or methoxy ethyl methacrylate. The backing material may also be comprised of a thermoplastic polyurethane film. Alternatively the backing material may be comprised of a microporous film of plasticized polyvinyl chloride or nonwoven fabric.

Another embodiment of the invention pertains to the use of a semi-permeable film or membrane dressing to reduce the transmission of HSV-1 or -2 or VZV. Herpetic wounds may be the result of an infection from HSV-1 or -2 or VZV.

Also presented herein are exemplary systems and methods for dressing viral lesions, and particularly for treating herpetic lesions, with a herpes dressing. An exemplary system includes a flexible treatment bandage system for treating a viral lesion having a flexible treatment layer configured for transparency, permeability to oxygen, impermeability to water and anchoring the flexible treatment layer around the viral lesion. According to various embodiments, a flexible treatment bandage system may further comprise an opposing side of the flexible treatment layer comprising a center area and a perimeter, the perimeter configured for weakly affixing to a supportive layer.

Other embodiments may comprise a supportive layer configured for weakly affixing to the perimeter of the opposing side of the flexible treatment layer for supporting the flexible treatment layer during affixing of the flexible treatment layer around the treatment area, and for resisting buckling of the flexible treatment layer during affixing of the flexible treatment layer. In some embodiments, the supportive layer may be a tab weakly affixed to the end of the flexible treatment layer. According to some embodiments, the exemplary system may further comprising a wrapping layer configured for weakly affixing to the supportive layer.

An exemplary method comprises anchoring a flexible treatment layer configured for transparency, permeability to oxygen, and impermeability to water, around a viral lesion. Other methods may further comprise removing a supportive layer configured for weakly affixing to a flexible treatment layer for supporting the flexible treatment layer during affixing of the flexible treatment layer around the viral lesion, and for resisting buckling of the flexible treatment layer during affixing of the flexible treatment layer around the viral lesion.

One aspect of the invention provides a method of treating a herpetic lesion (such as lesions caused by, e.g., herpes simplex 1, herpes simplex 2, or herpes zoster) including the step of applying a semi-permeable dressing to cover said wound for a sufficient time to heal said lesion. In some embodiments, the semi-permeable dressing is water vapor permeable and, optionally, liquid water impermeable and, optionally, oxygen permeable.

In some embodiments, the semi-permeable dressing has an adhesive and a backing material. The adhesive may be made of at least one of polyvinyl ethyl ether, (methyl vinyl ether), polymethyl vinyl ether, and acrylic ester. In some embodiments the backing material may be made of a copolymer made by copolymerizing a hydroxy alkyl acrylate or methacrylate with an alkoxy alkyl acrylate or methacrylate and optionally with a minor amount of a further monomer. In some embodiments the backing material may be made of a homopolymer of ethoxy ethyl methacrylate or methoxy ethyl methacrylate or a copolymer of methyl methacrylate with ethoxy methacrylate or methoxy ethyl methacrylate. In some embodiments the backing material may be made of a thermoplastic polyurethane film. In some embodiments the backing material may be made of a microporous film of plasticized polyvinyl chloride or nonwoven fabric. The dressing may also include an effective amount of medication for treatment of the viral lesion.

Another aspect of the invention provides a method of reducing the likelihood of transmitting herpes, including the step of applying a semi-permeable dressing to cover a herpetic wound of a subject. The dressing may be made of a semi-permeable film and may have an adhesive and a backing.

Yet another aspect of the invention provides a dressing assembly configured to treat a herpetic lesion including a semi-permeable material sized and shaped to cover a herpetic lesion on human tissue. Some embodiments also have an adhesive material and a backing material. The semi-permeable material may be water vapor permeable and liquid water impermeable and may be oxygen permeable. The dressing may also have an effective amount of medication for treatment of the viral lesion.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 shows a dressing assembly according to one embodiment of the invention in use to treat a viral lesion.

FIG. 2 shows a dressing assembly according to another embodiment of the invention.

FIG. 3 shows a dressing assembly according to yet another embodiment of the invention.

FIG. 4 is an exploded view of a dressing assembly according to still another embodiment of the invention.

FIG. 5 is an exploded view of a dressing assembly according to yet another embodiment of the invention.

FIG. 6 is an exploded view of a dressing assembly according to still another embodiment of the invention.

### DETAILED DESCRIPTION OF THE INVENTION

One embodiment of the invention uses a semi-permeable membrane dressing to inhibit the transmission of herpes. Another embodiment of the invention pertains to the prevention of secondary bacterial infections of the herpetic wound by providing a barrier to bacteria.

One aspect of the invention employs a thin, flexible, semi-permeable membrane as a dressing for a herpetic lesion. The membrane may be circular or any other suitable shape and may have a diameter of approximately 1-3 cm or other size as appropriate for the size of the lesion and the shape of the tissue surrounding the lesion. For example, a large dressing covering an entire affected area (e.g., diameters of a foot or more) may also be used. The membrane may be a polyurethane material such as those disclosed in U.S. Pat. No. 3,645,835. Such dressings have been used in the past on burns and are sold under the trademark TEGADERM® by The 3M Company, BIOCLUSIVE® by Johnson and Johnson; and OP-SITE®E by Smith and Nephew Inc. Such dressings are water vapor permeable to allow the skin to breathe, but are impermeable to liquid. These dressings are formed from a strong, highly flexible polyurethane material coated with a hypoallergenic, water resistant, polyacrylate adhesive. They are sterile, transparent, impenetrable by water and bacteria, but permeable to moisture vapor and oxygen.

The dressing of this invention may have an adhesive layer formed from, e.g., an acrylate polymer adhesive. For example, the Tegaderm dressing adhesive material, is aggressively tacky, pressure sensitive, and relatively nonirritating to the skin in comparison with plasticized rubber-resin compositions.

In some embodiments, the adhesive used on the dressing membrane is unaffected by water; i.e., it does not dissolve or soften or swell or lose its adhesion when exposed to water. The dressing membrane adhesive may also be permeable to water vapor and to oxygen. There are a number of adhesives suited to such application including acrylate adhesives, vinylacrylate adhesives and polyvinyl ether adhesives.

The membrane-adhesive combination of this invention is permeable to water vapor. This permeability allows evaporation of perspiration from the skin beneath the membrane helping the skin to remain dry and healthy and helping to minimize bacterial growth on the skin. The membrane-adhesive combination of this invention is also permeable to oxygen. This permeability allows oxygen to reach the skin, an important factor in promoting healing of any lesions on the skin and in promoting the bacteria fighting mechanisms of the skin. The membrane-adhesive combination of this invention is impermeable to bacteria.

In one embodiment of this invention the membrane is a continuous material made of polyurethane elastomer which can be manufactured in a manner which provides the desired thinness, flexibility, stretchability, and permeability to water vapor and oxygen. A preferred embodiment of the current invention is adhesive coated membranes having these desirable properties of thinness, low modulus of elasticity, and permeability. The membranes used in accordance with this invention have a thickness generally less than about ten thousandths and preferably less than about three thousandths of an inch (0.003).

The skin of the oral and genital areas can readily stretch. It follows that a dressing which is applied to the skin should also be able to be stretched by the attached skin in any direction and should be able to do so without exerting undue tugging force on the skin, which would be uncomfortable and tend to break the bond on the skin. The membranes used in this invention have sufficient stretchiness that it takes no more than 1.6 pounds/inch to elongate them substantially elastically by 20% and preferably less than 0.6 pounds/inch for 20% elongation. Where reference is made to the force required to elongate the dressing it is intended that such measurements are made by generally following ASTM Standard D882-02.

The average body loss of water through the skin, excluding visible sweat, is reported to be in the region of 250 g/m2/24 hrs, with areas such as the palms of the hands and soles of the feet having a higher water loss in the region of 500 g/m2/24 hrs. Thus, it follows that to allow the skin to continue its normal water output a dressing should have a water vapor permeability of at least 300 g/m2/24 hrs/40° C./80% relative humidity, with a water vapor permeability of at least 500 g/m2/24 hours/40° C./80% relative humidity being preferable considering that during hot weather and exercise the skin will need to get rid of additional perspiration.

Where reference is made to water vapor permeability it is intended that such measurements are made using the Payne cup method, carried out as follows. Ten milliliters of distilled water are added to the cup. A 1¼ inch diameter sample of the material to be tested is clamped above the opening from the cup. Where an adhesive is being tested this should first be coated onto a highly permeable backing for support. The arrangement is then placed in an air-circulating oven at temperatures of 40° C. and relative humidity of 20% for 24 hours. There is, therefore, a difference between the relative humidity inside the cup and the relative humidity outside the cup. The loss of water from the cup is found by weighing. The water vapor permeability is expressed as g/m2/24 hrs/40° C./80% relative humidity for the particular material.

A further important feature of the present invention is the use of a dressing which allows the skin to breathe. In order for the skin's healing mechanisms to function properly and in order to thwart the growth of anaerobic bacteria on the skin it is important for the skin to receive an adequate supply of oxygen. In the present invention it is possible to secure the full effect of the oxygen and water vapor permeability to contribute to the health and comfort of the skin. Where reference is made to water vapor permeability it is intended that such measurements are made in accordance with ASTM Test Method No. F1249-06. Where reference is made to oxygen permeability it is intended that such measurements are made in accordance with ASTM Test Method No. D3985-05.

### EXAMPLE 1

Tegaderm dressing is applied to a herpes lesion at the first sign of the lesion with 3 mm surrounding edge if applied to the oral area (e.g., the patient's lip) or 4 mm if applied to the genital area (e.g., penile skin, perineum, or labia). If the Tegaderm dressing falls off, then it is immediately replaced. According to the above procedure, ten patients are treated. By the fourth day the Tegaderm is removed and all the treated wounds are re-epithelialized

By contrast, under standard herpes therapies, at the first indication of a herpes outbreak (often a tingling, itching, or burning sensation) Valtrex is given orally to the patient. Valtrex is administered to the patient twice a day for five days at dosage of 250 mg, 500 mg or 1 gram. At the end of the treatment sores remain and do not heal for an additional 3-4 days.

As seen with the above example, covering the herpetic lesion with Tegaderm dressing hastens the healing time of herpetic wounds by at least two-fold.

### EXAMPLE 2

To determine a Tegaderm dressing's effectiveness as a viral barrier, it is subjected to the ASTM Test Method No. F1671 (Standard Test Method for Resistance of Materials Used in Protective Clothing to Penetration by Blood-Borne Pathogens Using Phi-X 174 Bacteriophage Penetration as a Test System). The test is used to measure the resistance of materials “to penetration by blood-borne pathogens using a surrogate microbe under conditions of continuous liquid contact.” Specifically, this test method utilizes Hepatitis (B and C) and Human Immunodeficiency Viruses for modeling the viral penetration of the Tegaderm dressing. In addition, Phi-X 174 Bacteriophage, which is one of the smallest known viruses (0.025 μm in diameter) and is also much smaller than bacteria, is also used. Phi-X 174 Bacteriophage provides a greater challenge to the test material and therefore, these results indicate that the material is resistant to viral and bacterial penetration. Pass or fail determinations are based on the detection of viral penetration. Both Tegaderm and Tegaderm HP dressings pass the testing criteria described above; that is viral penetration is not detected. Therefore, these dressings and the films made from these dressings are resistant to viral penetration and behave as viral and bacterial barriers. Accordingly it is likely that Tegaderm will prevent spread of HSV, because HSV is one of the largest known viruses.

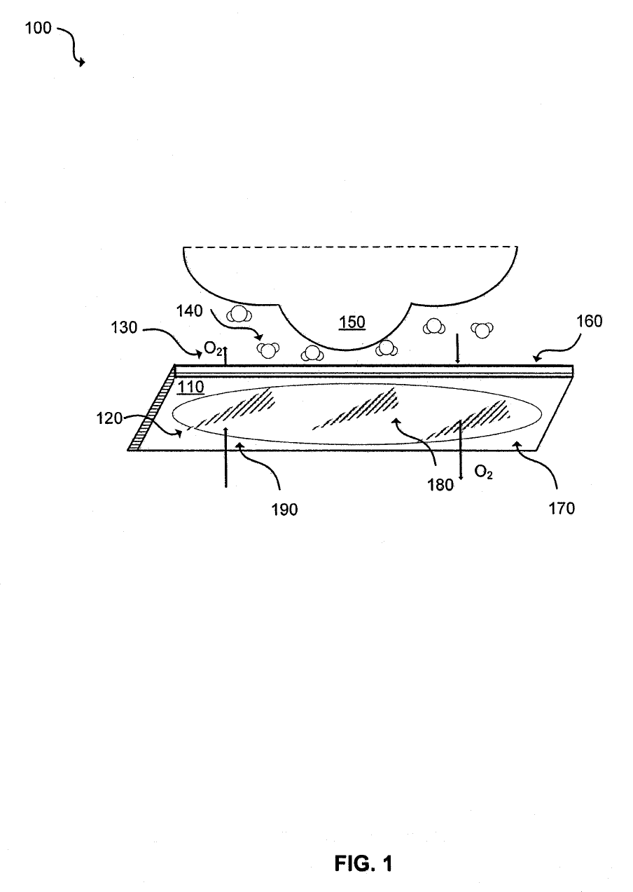

FIG. 1 shows an exemplary environment 100 where embodiments of the herpes dressing may be practiced. In various embodiments, an exemplary system comprises a flexible treatment layer 110 configured for transparency (as illustrated by the triangular cross-hatched areas 120), permeability to oxygen (as illustrated by O₂ arrows 130, impermeability to liquid water (as illustrated by schematic representations of water molecules 140), and for anchoring the flexible treatment layer 110 around the viral lesion 150. In some embodiments, the viral lesion is a herpetic lesion.

According to various embodiments, surface 160 may provide the configuration of the flexible treatment layer 110 for anchoring the flexible treatment layer 110 around the viral lesion 150. In some embodiments, the surface 160 may be adhesive. In some embodiments, the surface 160 may be treated for adhesion and abhesion to weakly affix around the viral lesion 150.

In other embodiments, the exemplary system 110 may further comprise an opposing side 170 further comprising a center area 180 and a perimeter 190, the perimeter configured for weakly affixing to a supportive layer. The perimeter 190 may adhesive. In some embodiments, the perimeter 190 may be treated for adhesion and abhesion to weakly affix to a supportive layer.

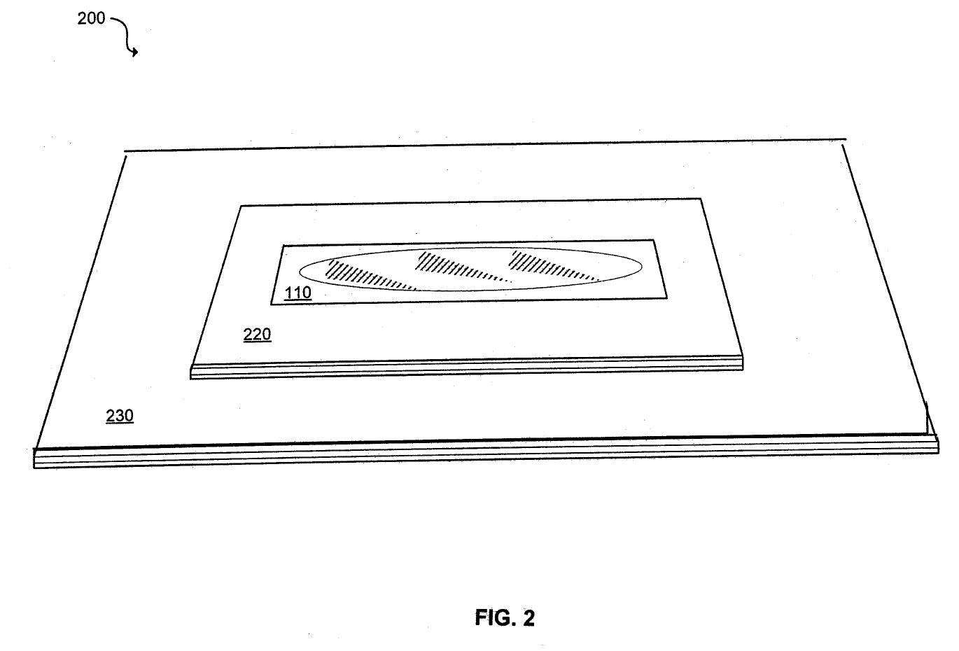

In some embodiments, the dressing may include a supportive layer for supporting the flexible treatment layer and a wrapping layer. The supportive layer permits applying the flexible treatment layer without touching the lesion. FIG. 2 shows an exemplary system 200 of the herpes dressing of this invention for treating a viral lesion. In one embodiment, the exemplary system has a supportive layer 220 configured for weakly affixing to the perimeter of the opposing side of the flexible treatment layer 110 for supporting the flexible treatment layer during affixing of the flexible treatment layer around the treatment area, and for resisting buckling of the flexible treatment layer during affixing of the flexible treatment layer. In some embodiments, the flexible. treatment layer 110 may be sandwiched between the supportive layer 220 and the wrapping layer 230.

In another embodiment, the herpes dressing further comprises a wrapping layer 230 configured for weakly affixing to the supportive layer. The wrapping layer 230 is weakly affixed to the supportive layer 230 to aid in anchoring the flexible treatment layer around the viral lesion without touching the viral lesion.

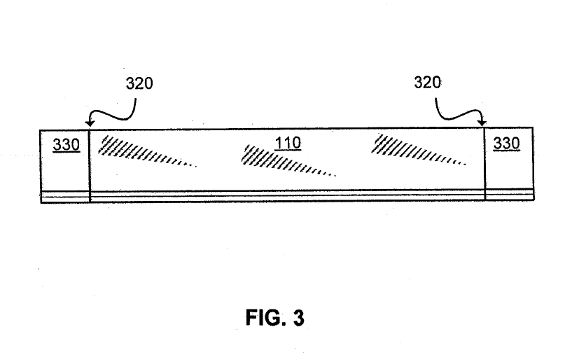

FIG. 3 shows one exemplary system according to some embodiments. In these embodiments, a flexible treatment bandage system for treating a viral lesion may comprise a flexible treatment layer 110 further comprising a treatment side configured for transparency, permeability to oxygen, impermeability to water, and anchoring the flexible treatment layer around the viral lesion; an end 320 configured for weakly affixing to a support tab; and a support tab 330 weakly affixed to an end of the flexible treatment layer for supporting the flexible treatment layer during affixing of the flexible treatment layer around the treatment area and for resisting buckling of the flexible treatment layer during affixing of the flexible treatment layer.

In an exemplary method for treating a viral lesion (such as a herpes lesion), a flexible treatment layer configured for transparency, permeability to oxygen and impermeability to water is anchored around a viral lesion. A supportive layer is then removed. The supportive layer is configured for weakly affixing to the flexible treatment layer to support the flexible treatment layer during affixing of the flexible treatment layer around the viral lesion. The supportive layer also resists buckling of the flexible treatment layer during affixing of the flexible treatment layer around the viral lesion. The supportive layer may be e.g., semi-circular or any other suitable shape. Some embodiments may also have a wrapping layer configured for weakly affixing to the supportive layer to aid affixing the flexible treatment layer around the viral lesion without touching the viral lesion. In some embodiments, the supportive layer overlaps to aid securing the flexible treatment layer. In some embodiments, the supportive layer is curved to aid removal of the supportive layer by pulling around the treatment layer. This may have the advantage of not pulling the treatment layer off the skin, and removing the supportive layer may be performed with one hand.

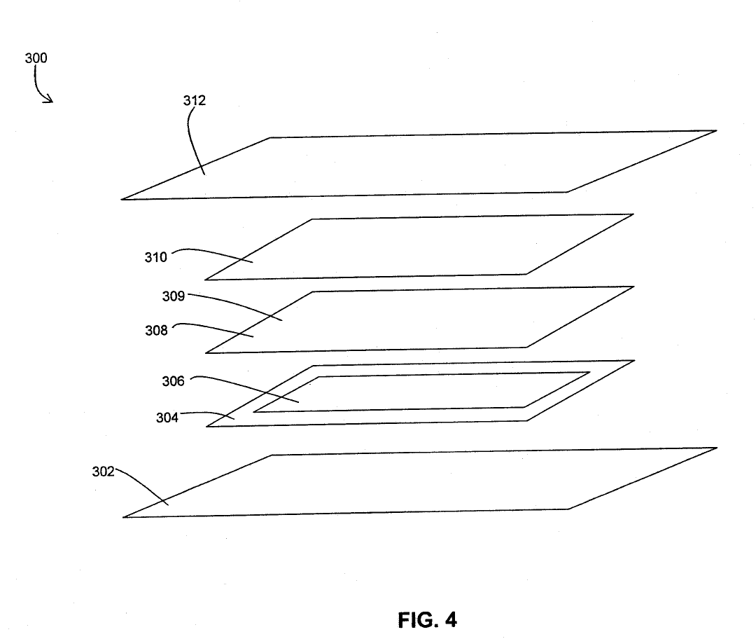

FIG. 4 shows an embodiment of the herpetic lesion dressing of this invention. Enclosed within upper and lower outer wrapping layers 312 and 302 is a dressing having a supportive frame 304 surrounding an opening 306; a semi-permeable dressing layer 308 having an adhesive 309 on its upper surface; and a protective barrier 310. Protective barrier 310 is applied to the underside of wrapper layer 312 so that it is removed when the wrapping is removed. Dressing layer 308 is clear, oxygen permeable and liquid water impermeable. The adhesive upper side of layer 308 is applied directly to the herpetic lesion after removal of layers 310 and 312. There may also be adhesive on the upper side of frame 304 to lightly affix it to the under (non-sticky) side of dressing layer 308. Adhesive may also lightly affix wrapper layer 302 to the underside of frame layer 304.

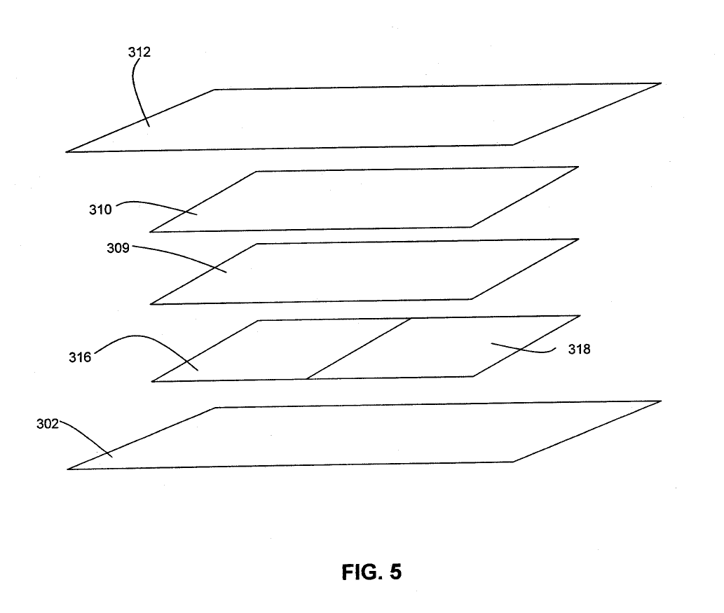

FIG. 5 shows an alternative to frame layer 304. In this embodiment, the frame is formed in two halves 316 and 318, each of which can be removed separately from dressing layer 308.

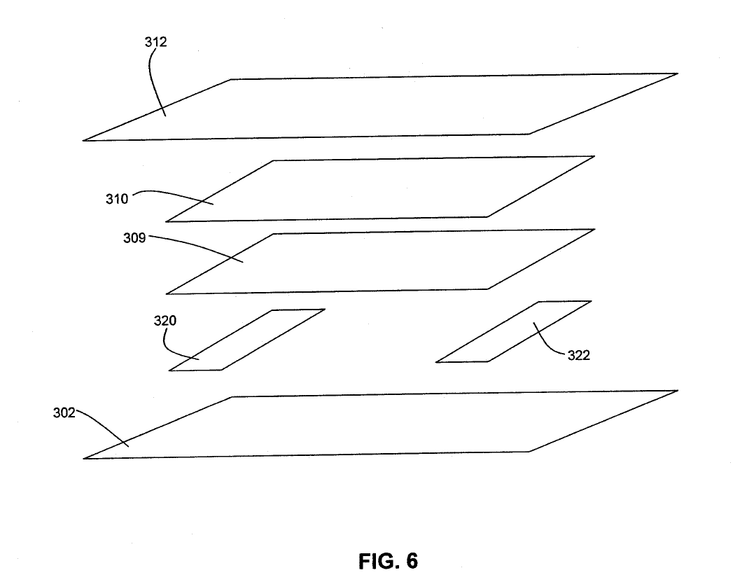

FIG. 6 shows another alternative in which the frame layer is replaced with two anchoring tabs 320 and 322 affixed to opposite ends of dressing layer 308.

While the preferred embodiment of the invention has been illustrated and described, it will be appreciated that various changes can be made therein without departing from the spirit and scope of the invention. Therefore the scope of the invention is to be determined from the following claims and equivalents thereof.

What is claimed is:

1. A method of treating a herpetic lesion comprising applying a semi-permeable dressing to cover said wound for a sufficient time to heal said lesion.

2. The method of claim 1 wherein a source of said herpetic lesion is selected from the group consisting of herpes simplex 1, herpes simplex 2, or herpes zoster.

3. The method of claim 1 wherein the semi-permeable dressing is water vapor permeable.

4. The method of claim 1 wherein the semi-permeable dressing is water vapor permeable and liquid water impermeable.

5. The method of claim 1 wherein the semi-permeable dressing is oxygen permeable.

6. The method of claim 1 wherein said semi-permeable dressing comprises an adhesive and a backing material.

7. The method of claim 6 wherein said adhesive is comprised of at least one of polyvinyl ethyl ether, (methyl vinyl ether), polymethyl vinyl ether, and acrylic ester.

8. The method of claim 6 wherein said backing material is comprised of a copolymer made by copolymerizing a hydroxy alkyl acrylate or methacrylate with an alkoxy alkyl acrylate or methacrylate and optionally with a minor amount of a further monomer.

9. The method of claim 6 wherein said backing material is comprised of a homopolymer of ethoxy ethyl methacrylate or methoxy ethyl methacrylate or a copolymer of methyl methacrylate with ethoxy methacrylate or methoxy ethyl methacrylate.

10. The method of claim 6 wherein said backing material is comprised of a thermoplastic polyurethane film.

11. The method of claim 6 wherein said backing material is comprised of a microporous film of plasticized polyvinyl chloride or nonwoven fabric.

12. The method of claim 6 wherein the dressing comprises an effective amount of medication for treatment of the viral lesion.

13. A method of reducing the likelihood of transmitting herpes comprising applying a semi-permeable dressing to cover a herpetic wound of a subject.

14. The method of claim 13 wherein said dressing comprises a semi-permeable film.

15. The method of claim 13 where said dressing comprises an adhesive and a backing.

16. A dressing assembly configured to treat a herpetic lesion, the dressing assembly comprising a semi-permeable material sized and shaped to cover a herpetic lesion on human tissue.

17. The dressing assembly of claim 16 further comprising an adhesive material and a backing material.

18. The dressing assembly of claim 16 wherein the semi-permeable material is water vapor permeable and liquid water impermeable.

19. The dressing assembly of claim 18 wherein the semi-permeable material is oxygen permeable.

20. The dressing assembly of claim 16 further comprising an effective amount of medication for treatment of the viral lesion.
